(19) United States
(12) Patent Application Publication
(10) Pub. No.: US 2019/0298763 A1
(43) Pub. Date: Oct. 3, 2019

(54) USE OF DNA NETOSIS TO DELIVER TRAIL FOR CANCER THERAPY

(71) Applicant: Vanderbilt University, Nashville, TN (US)

(72) Inventors: Michael KING, Nashville, TN (US); Thong CAO, Nashville, TN (US)

(21) Appl. No.: 16/364,903

(22) Filed: Mar. 26, 2019

Related U.S. Application Data
(60) Provisional application No. 62/648,642, filed on Mar. 27, 2018.

(51) Int. Cl. A61K 35/15; C07K 14/525; A61K 45/06; A61K 38/20; A61K 38/06; A61K 9/127; A61K 9/06; C07K 14/435; A61P 35/00

(52) U.S. Cl. CPC A61K 35/15; C07K 14/525; A61K 45/06; A61K 38/2053; C07K 2319/60; A61K 9/127; A61K 9/06; C07K 14/43504; A61P 35/00; A61K 38/06

(57) ABSTRACT

The present disclosure is directed to the TNF-related apoptosis inducing ligand (TRAIL) fusions with positively charged proteins, neutrophils engineered to express and secrete such fusions in the context of neutrophil extracellular traps, and methods of use thereof in the treatment of cancer.

### PRIORITY CLAIM

This application claims benefit of priority to U.S. Provisional Application Ser. No. 62/648,642, filed Mar. 27, 2018, the entire contents of which are hereby incorporated by reference.

### BACKGROUND

### 1. Field of the Disclosure

The present disclosure relates generally to the fields of medicine, oncology, molecular biology and immunology. More particular, the disclosure relates to TNF-related apoptosis inducing ligand (TRAIL) fusions with positively charged proteins, and methods of use in the treatment of cancer by targeting TRAIL fusions to neutrophil extracellular trap (NET) DNA.

### 2. Background

Neutrophils play a significant role in all stages of tumorigenesis from the initial genotoxic insult to metastasis to distant organs. Chronic inflammation drives neutrophils to release mutagenetic agents including reactive oxygen species (ROS) and hypochlorous acid (HOC1) that induce DNA damage and mutagenicity on surrounding cells (Godschalk et al., 2009; Atkins et al., 2009; Jurk et al., 2015). Neutrophils make up a significant percentage of white blood cells that infiltrate the tumor microenvironment (Gregory & Houghton, 2011). Here, infiltrating neutrophils continue to promote tumor development by secreting pro-inflammatory and pro-angiogenesis chemokines and cytokines such as matrix metallopeptidase 9 (MMP9) and interleukin 6 (IL-6) (Nozawa et al., 2006; Albelda et al., 2009; Thacker et al., 2010). Tumor metastasis through hematogenous dissemination involves circulating tumor cells (CTCs) shedding from the primary tumor site and reaching distant organs via the circulatory system. In a recent study, neutrophils have been found to support CTC survival during hematogenous dissemination (Szcerba et al., 2019). Furthermore, neutrophils have been identified as the main driver in establishing the pre-metastatic microenvironment in several mouse breast cancer models (Wculek & Malanchi, 2015).

Netosis is an antimicrobial response by neutrophils where the cells release condensed DNA fibers decorated with positively charged antimicrobial proteins called neutrophil extracellular traps (NETs) into the extracellular space (Brinkmann et al., 2004). Circulating tumor cells (CTCs) found in the pulmonary vasculature secrete a high level of cytokine IL-8, a netosis agonist, which attracts neutrophils and increases their sensitivity for netosis (Hidalgo et al., 2015). Lartique et al. observed that NETs can sequester CTCs, however the trapped CTCs do not die, but instead survive and are able to use NETs as a physical bridge to increase their metastatic potential (Cool-Lartique et al., 2013).

TNF-related apoptosis-inducing ligand (TRAIL), is a protein functioning as a ligand that induces the process of cell death called apoptosis. TRAIL is a cytokine that is produced and secreted by most normal tissue cells. It causes apoptosis primarily in tumor cells, by binding to certain death receptors. As such, TRAIL and its receptors have been explored as reagents or targets in in a variety of anti-cancer therapeutics going back to the mid-1990s, such as the antibody Mapatumumab, which targets TRAIL Receptor 1. However, for the most part, these therapeutic approaches have yet to yield satisfactory result. Therefore, improved methods of using TRAIL and other proapoptotic molecuels in cancer therapy are needed.

### SUMMARY

Thus, in accordance with the present disclosure, there is provided a method inhibiting cancer in a subject comprising administering to said subject a first dose of a neutrophil engineered to express TNF-related apoptosis inducing ligand (TRAIL) fused to a positively-charged peptide or protein segment. The engineered neutrophil may have been transformed with an exogenous mRNA encoding the TRAIL fusion or transformed with an expression vector encoding the TRAIL fusion. The expression vector may be a viral expression vector, or a non-viral expression vector. The method may further comprise co-administering one or more agents that promote netosis, such as fmlp or IL-8, which may be delivered liposomes or hydrogels, or in pH sensitive liposomes or hydrogels. Alternatively, the method may comprise transfecting a pluripotent stem cell or a neutrophil in said cell with a construct that expresses TNF-related apoptosis inducing ligand (TRAIL) fused to a positively-charged peptide or protein segment. The construct may be administered through a route capable of targeting the subject's bone marrow.

In alternative embodiments, the neutrophil is engineered to produce a distinct mRNA for another pro-apoptotic molecule such as FasL, p28, or arginine deaminase, fused to a positively-charged peptide or protein segment. The engineered neutrophil may have been transformed with an exogenous mRNA or transformed with an expression vector encoding the mRNA. The expression vector may be a viral expression vector, or a non-viral expression vector. The method may further comprise co-administering one or more agents that promote netosis, such as fmlp or IL-8, which may be delivered liposomes or hydrogels, or in pH sensitive liposomes or hydrogels. Alternatively, the method may comprise transfecting a pluripotent stem cell or a neutrophil in said cell with a construct that expresses FasL, p28 or arginine deaminase fused to a positively-charged peptide or protein segment. The construct may be administered through a route capable of targeting the subject's bone marrow.

The engineered neutrophil may be administered systemically. The engineered neutrophil may be administered local or region to a cancer site, or into a tumor bed or into tumor vasculature. The method may further comprise administering to said subject a second dose of said engineered neutrophil. The neutrophils can be transfected outside of the body, or within the recipient's body. The engineered neutrophils can be created by transfecting pluripotent hematopoetic stem cells, living in the bone marrow, which differentiate into mature neutrophils. The positively-charged protein or peptide may be a protein with electrostatic charge ranging from +4 to +24, or +4 to +20. The positively-charged protein or peptide may be green fluorescent protein or a variant thereof that retains fluorescent activity. The positively-charged protein may be 100-500 residues, such as 100 residues, 125 residues, 150 residues, 175 residues, 200 residues, 250 residues, 300 residues, 350 residues, 400 residues, 450 residues, or 500 residues. The peptide is less than 100 resides, such as 10 residues, 20 residues, 30 residues, 40 residues, 50 residues, 60 residues, 70 residues, 80 residues, or 90 residues, including 10-90 residues.

The method may further comprise administering to said subject a second cancer therapy, such as chemotherapy, radiotherapy, immunotherapy, surgery or hormonal therapy. The neutrophil may be autologous to said subject, or not autologous to said subject. The subject may be a non-human animal or a human. The cancer may be recurrent, metastatic and/or or drug resistant. Inhibiting cancer may comprise inducing apoptosis in cells of said cancer.

Also provided is a neutrophil engineered to express TNF-related apoptosis inducing ligand (TRAIL) fused to a positively-charged peptide or protein segment. The neutrophil may have been transformed with an exogenous mRNA encoding the TRAIL fusion or transformed with an expression vector encoding the TRAIL fusion. The expression vector may be a viral expression vector, or a non-viral expression vector.

The positively-charged protein or peptide may be a protein with electrostatic charge ranging from +4 to +24, or +4 to +24. The positively-charged protein or peptide may be green fluorescent protein or a variant thereof that retains fluorescent activity. The positively-charged protein may be 100-500 residues, such as 100 residues, 125 residues, 150 residues, 175 residues, 200 residues, 250 residues, 300 residues, 350 residues, 400 residues, 450 residues, or 500 residues. The peptide is less than 100 resides, such as 10 residues, 20 residues, 30 residues, 40 residues, 50 residues, 60 residues, 70 residues, 80 residues, or 90 residues, including 10-90 residues. The positively-charged peptide or protein may be attached to the N-terminus of TRAIL. The method may further comprise engineering a neutrophil to produce enhanced netosis.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. (FIG. 1A) Human neutrophils electroporated with GFP-TRAIL mRNA express TRAIL proteins 4 h post electroporation. (FIG. 1B) 24 h post electroporation, 900 nM of PMA was added to neutrophils to induce netosis.

FIGS. 2A-B. Representative plots of flow cytometry of COLO 205 cell line coculture overnight with transfected neutrophils and assayed for apoptosis using Annexin V/PI staining. The x-axis is FITC-Annexin V and the y-axis is PI. FIG. 2A is the control assay; FIG. 2B is the test assay.

FIGS. 3A-E. Neutrophils undergo spontaneous NETosis in the presence of tumor cells. (FIG. 3A) cfDNA plasma DNA level of normal donors (ND) and cancer patients (PAT). (FIG. 3B) Confocal images of neutrophils underwent NETosis in conditioned media. (CTL)—cell cultured normal cultured medium, (COLO-205 and SW-620 are condition media collected after 48 h from these indicated cell lines). (FIG. 3C) NETosis level of neutrophils after culturing in conditioned media for 24 h. (FIGS. 3D-E) Extracellular Tumor-derived IL-8 level in media. Reparixin concentration (20 μg/mL).

FIGS. 4A-F. Engineered neutrophils express supercharged eGFP-TRAIL on NETs during NETosis. (FIG. 4A) Schematic of the insertion site, AAVS1, on chromosome 19. Cartoon representation of eGFP-TRAIL chimeric protein. Surface charge of eGFP ranging from −4 to +36 (Red—negative charge, Blue—positive charge. (FIG. 4B) DNA sequencing result of genomic DNA isolated from cells positive for eGFP-TRAIL (SEQ ID NO: 1). (FIG. 4C) relative mRNA level of endogenous (enTRAIL) and exogenous (exTRAIL) TRAIL levels. (FIG. 4D) Flow cytometry of cells expressing eGFP-TRAIL 24 h after nucleofection. (FIG. 4E) Confocal images of NETs decorated with eGFP-Trail (Blue—DAPI stain, Green—eGFP-TRAIL. (FIG. 4F) Immuno-gold SEM images of neutrophil expressing eGFP-decorated NETs (White arrows—eGFP-TRAIL).

FIGS. 5A-C. Supercharged eGFP-TRAIL expressing neutrophils trap and destroy tumor cells during NETosis. (FIG. 5A) Representative flow cytometry result of COLO-205 cells co-cultured with eGFP-TRAIL expressing neutrophils for 24 h and stained for apoptosis and necrosis using Annexin V and Propodium Iodide, respectively. (FIG. 5B) Cell viability quantification of indicated cancer cell lines co-cultured with wildtype neutrophils or positive for eGFP-TRAIL (Tf). (FIG. 5C) Immuno-gold SEM images of indicated cancer cells co-cultured with neutrophils.

### DESCRIPTION OF ILLUSTRATIVE EMBODIMENTS

As discussed above, NETs interact with CTCs in a unique fashion. The inventors surmised that this interaction could be exploited as a novel cancer therapeutic platform to combat metastasis due to their sustained proximity to metastasizing cancer cells. TNF-related apoptosis-inducing ligand (TRAIL) is a protein that selectively binds and induces apoptosis in cancer cells overexpressing death receptors while sparing healthy cells. The inventors have developed multiple TRAIL delivery platforms to target metastasizing tumor cells. In this study, the inventors expanded the existing technologies by combining the CTC-sequestering potential of NETs and the anti-tumoral capability of TRAIL. By re-engineering neutrophils to express NETs decorated with charged TRAIL, they demonstrate that neutrophils releasing TRAIL-decorated NETs in the presence of tumor cells ensnare these cells and induce TRAIL-mediated apoptosis. These and other aspects of the disclosure are described in detail below.

### I. NEUTROPHILS

Neutrophils (also known as neutrocytes) are the most abundant type of granulocytes and the most abundant (40% to 70%) type of white blood cells in most mammals. They form an essential part of the innate immune system. Their functions vary in different animals. They are formed from stem cells in the bone marrow. They are short-lived and highly motile, as they can enter parts of tissue where other cells/molecules cannot. Neutrophils may be subdivided into segmented neutrophils and banded neutrophils (or bands). They form part of the polymorphonuclear cells family (PMNs) together with basophils and eosinophils.

The name “neutrophil” derives from staining characteristics on hematoxylin and eosin (H&E) histological or cytological preparations. Whereas basophilic white blood cells stain dark blue and eosinophilic white blood cells stain bright red, neutrophils stain a neutral pink. Normally, neutrophils contain a nucleus divided into 2-5 lobes.

Neutrophils are a type of phagocyte and are normally found in the bloodstream. During the beginning (acute) phase of inflammation, particularly as a result of bacterial infection, environmental exposure, and some cancers, neutrophils are one of the first-responders of inflammatory cells to migrate towards the site of inflammation. They migrate through the blood vessels, then through interstitial tissue, following chemical signals such as Interleukin-8 (IL-8), C5a, fMLP, Leukotriene B4 and H₂O₂ in a process called chemotaxis. They are the predominant cells in pus, accounting for its whitish/yellowish appearance.

Neutrophils are recruited to the site of injury within minutes following trauma and are the hallmark of acute inflammation; however, due to some pathogens being indigestible, they can be unable to resolve certain infections without the assistance of other types of immune cells.

When adhered to and spread on a surface, neutrophil granulocytes have an average diameter of 12-15 micrometers (μm) in peripheral blood smears. In suspension, human neutrophils have an average diameter of 8.85 μm.

With the eosinophil and the basophil, neutrophils form the class of polymorphonuclear cells, named for the nucleus' multilobulated shape (as compared to lymphocytes and monocytes, the other types of white cells). The nucleus has a characteristic lobed appearance, the separate lobes connected by chromatin. The nucleolus disappears as the neutrophil matures, which is something that happens in only a few other types of nucleated cells. In the cytoplasm, the Golgi apparatus is small, mitochondria and ribosomes are sparse, and the rough endoplasmic reticulum is absent. The cytoplasm also contains about 200 granules, of which a third are azurophilic. Neutrophils are sexually dimorphic. Neutrophils from women exhibit a small additional X chromosome structure, known as a “neutrophil drumstick.”

Neutrophils will show increasing segmentation (many segments of the nucleus) as they mature. A normal neutrophil should have 3-5 segments. Hypersegmentation is not normal but occurs in some disorders, most notably vitamin B12 deficiency. This is noted in a manual review of the blood smear and is positive when most or all of the neutrophils have 5 or more segments.

Neutrophils are the most abundant white blood cells in humans (approximately 10¹¹ are produced daily); they account for approximately 50-70% of all white blood cells (leukocytes). The stated normal range for human blood counts varies between laboratories, but a neutrophil count of 2.5-7.5×10⁹/L is a standard normal range. People of African and Middle Eastern descent may have lower counts, which are still normal. A report may divide neutrophils into segmented neutrophils and bands. When circulating in the bloodstream and inactivated, neutrophils are spherical. Once activated, they change shape and become more amorphous or amoeba-like and can extend pseudopods as they hunt for antigens.

Neutrophils have a preference to engulf refined carbohydrates (from ingested glucose, fructose, sucrose, honey and orange juice) over bacteria. In 1973, researchers found that the neutrophil phagocytic capacity to engulf bacteria is affected when simple sugars are digested, and that fasting strengthens the neutrophils' phagocytic capacity to engulf bacteria. However, the digestion of normal starches has no effect. It was concluded that the function, and not the number, of phagocytes in engulfing bacteria was altered by the ingestion of sugars. In 2007 researchers at the Whitehead Institute of Biomedical Research found that given a selection of sugars, neutrophils engulf some types of sugar preferentially.

The average lifespan of inactivated human neutrophils in the circulation has been reported by different approaches to be between 5 and 90 hours. Upon activation, they marginate (position themselves adjacent to the blood vessel endothelium) and undergo selectin-dependent capture followed by integrin-dependent adhesion in most cases, after which they migrate into tissues, where they survive for 1-2 days.

Neutrophils are much more numerous than the longer-lived monocyte/macrophage phagocytes. A pathogen (disease-causing microorganism or virus) is likely to first encounter a neutrophil. Some experts hypothesize that the short lifetime of neutrophils is an evolutionary adaptation. The short lifetime of neutrophils minimizes propagation of those pathogens that parasitize phagocytes because the more time such parasites spend outside a host cell, the more likely they will be destroyed by some component of the body's defenses. Also, because neutrophil antimicrobial products can also damage host tissues, their short life limits damage to the host during inflammation. Neutrophils will be removed after phagocytosis of pathogens by macrophages. PECAM-1 and phosphatidylserine on the cell surface are involved in this process.

Neutrophils undergo a process called chemotaxis via amoeboid movement, which allows them to migrate toward sites of infection or inflammation. Cell surface receptors allow neutrophils to detect chemical gradients of molecules such as interleukin-8 (IL-8), interferon gamma (IFN-γ), C3a, C5a, and Leukotriene B4, which these cells use to direct the path of their migration.

Neutrophils have a variety of specific receptors, including ones for complement, cytokines like interleukins and IFN-γ, chemokines, lectins, and other proteins. They also express receptors to detect and adhere to endothelium and Fc receptors for opsonin.

In leukocytes responding to a chemoattractant, the cellular polarity is regulated by activities of small Rho guanosine triphosphatases (Rho GTPases) and the phosphoinositide 3-kinases (PI3Ks). In neutrophils, lipid products of PI3Ks regulate activation of Rho GTPases and are required for cell motility. They accumulate asymmetrically to the plasma membrane at the leading edge of polarized cells. Spatially regulating Rho GTPases and organizing the leading edge of the cell, PI3Ks and their lipid products could play pivotal roles in establishing leukocyte polarity, as compass molecules that tell the cell where to crawl.

It has been shown in mice that in certain conditions neutrophils have a specific type of migration behavior referred to as neutrophil swarming during which they migrate in a highly coordinated manner and accumulate and cluster to sites of inflammation.

Being highly motile, neutrophils quickly congregate at a focus of infection, attracted by cytokines expressed by activated endothelium, mast cells, and macrophages. Neutrophils express and release cytokines, which in turn amplify inflammatory reactions by several other cell types.

In addition to recruiting and activating other cells of the immune system, neutrophils play a key role in the front-line defense against invading pathogens. Neutrophils have three methods for directly attacking micro-organisms: phagocytosis (ingestion), degranulation (release of soluble anti-microbials), and generation of neutrophil extracellular traps (NETs), as discussed further below.

Neutrophils are phagocytes, capable of ingesting microorganisms or particles. For targets to be recognized, they must be coated in opsonins—a process known as antibody opsonization. They can internalize and kill many microbes, each phagocytic event resulting in the formation of a phagosome into which reactive oxygen species and hydrolytic enzymes are secreted. The consumption of oxygen during the generation of reactive oxygen species has been termed the “respiratory burst,” although unrelated to respiration or energy production.

The respiratory burst involves the activation of the enzyme NADPH oxidase, which produces large quantities of superoxide, a reactive oxygen species. Superoxide decays spontaneously or is broken down via enzymes known as superoxide dismutases (Cu/ZnSOD and MnSOD), to hydrogen peroxide, which is then converted to hypochlorous acid (HClO), by the green heme enzyme myeloperoxidase. It is thought that the bactericidal properties of HClO are enough to kill bacteria phagocytosed by the neutrophil, but this may instead be a step necessary for the activation of proteases.

Neutrophils also release an assortment of proteins in three types of granules by a process called degranulation. The contents of these granules have antimicrobial properties, and help combat infection:

| Granule type | Protein |
|---|---|
| Azurophilic granules | Myeloperoxidase, bactericidal/permeability-increasing protein (BPI), |
| (or “primary granules”) | defensins, and the serine proteases neutrophil elastase and cathepsin |
| | G |
| Specific granules | Alkaline phosphatase, lysozyme, NADPH oxidase, collagenase, |
| (or “secondary granules”) | lactoferrin, histaminase, and cathelicidin |
| Tertiary granules | Cathepsin, gelatinase and collagenase |

Low neutrophil counts are termed neutropenia. This can be congenital (developed at or before birth) or it can develop later, as in the case of aplastic anemia or some kinds of leukemia. It can also be a side-effect of medication, most prominently chemotherapy. Neutropenia makes an individual highly susceptible to infections. It can also be the result of colonization by intracellular neutrophilic parasites.

In alpha 1-antitrypsin deficiency, the important neutrophil enzyme elastase is not adequately inhibited by alpha 1-antitrypsin, leading to excessive tissue damage in the presence of inflammation—the most prominent one being pulmonary emphysema.

In Familial Mediterranean fever (FMF), a mutation in the pyrin (or marenostrin) gene, which is expressed mainly in neutrophil granulocytes, leads to a constitutively active acute-phase response and causes attacks of fever, arthralgia, peritonitis, and—eventually—amyloidosis.

Decreases in neutrophil function have been linked to hyperglycemia. Dysfunction in the neutrophil biochemical pathway myeloperoxidase as well as reduced degranulation are associated with hyperglycemia.

The Absolute neutrophil count (ANC) is also used in diagnosis and prognosis. ANC is the gold standard for determining severity of neutropenia, and thus neutropenic fever. Any ANC <1500 cells/mm3 is considered neutropenia, but <500 cells/mm³ is considered severe. There is also new research tying ANC to myocardial infarction as an aid in early diagnosis. Primary human neutrophils can be isolated as described previously in Mitchell & King (2012) and Lee et al. (2007). Whole peripheral blood can be obtained via venous needle injection from healthy human donors after informed consent. Neutrophils are separated by centrifugation at 480×g at 23° C. for 50 min in a Marathon 8 K centrifuge (Fisher Scientific, Pittsburgh, Pa.) using 1-Step Polymorphs (Accurate Chemical and Scientific Corporation, Westbury, N.Y.), and resuspended in Mg₂ and Cat-free HBSS to remove excess polymorph solution. Remaining red blood cells are then lysed hypotonically, and purified neutrophils are resuspended in Mg₂-free HBSS buffer with 0.5% human serum albumin, 10 mM HEPES, and 2 mM Ca₂. at a pH of 7.4 at a concentration of 0.5×10⁶ cells/mL.

### II. NEUTROPHIL EXTRACELLULAR TRAPS AND NETOSIS

Neutrophil extracellular traps (NETs) are networks of extracellular fibers, primarily composed of DNA from neutrophils, which bind pathogens. As noted above, neutrophils are the immune system's first-line of defense against infection and have conventionally been thought to kill invading pathogens through two strategies: engulfment of microbes and secretion of anti-microbials. In 2004, a novel third function was identified—formation of NETs. NETs allow neutrophils to kill extracellular pathogens while minimizing damage to the host cells. Upon in vitro activation with the pharmacological agent phorbol myristate acetate (PMA), Interleukin 8 (IL-8) or lipopolysaccharide (LPS), neutrophils release granule proteins and chromatin to form an extracellular fibril matrix known as NETs through an active process.

High-resolution scanning electron microscopy has shown that NETs consist of stretches of DNA and globular protein domains with diameters of 15-17 nm and 25 nm, respectively. These aggregate into larger threads with a diameter of 50 nm. However, under flow conditions, NETs can form much larger structures, reaching hundreds of nanometers in length and width.

Analysis by immunofluorescence corroborated that NETs contain proteins from azurophilic granules (neutrophil elastase, cathepsin G and myeloperoxidase), specific granules (lactoferrin), tertiary granules (gelatinase), and the cytoplasm; however, CD63, actin, tubulin and various other cytoplasmatic proteins are not present in NETs.

NETs disarm pathogens with antimicrobial proteins such as neutrophil elastase, cathepsin G and histones that have a high affinity for DNA. NETs provide for a high local concentration of antimicrobial components and bind, disarm, and kill microbes extracellularly independent of phagocytic uptake. In addition to their antimicrobial properties, NETs may serve as a physical barrier that prevents further spread of the pathogens. Furthermore, delivering the granule proteins into NETs may keep potentially injurious proteins like proteases from diffusing away and inducing damage in tissue adjacent to the site of inflammation.

More recently, it has also been shown that not only bacteria but also pathogenic fungi such as Candida albicans induce neutrophils to form NETs that capture and kill C. albicans hyphal as well as yeast-form cells. NETs have also been documented in association with Plasmodium falciparum infections in children.

While it was originally proposed that NETs would be formed in tissues at a site of bacterial/yeast infection, NETs have also been shown to form within blood vessels during sepsis (specifically in the lung capillaries and liver sinusoids). Intra-vascular NET formation is tightly controlled and is regulated by platelets, which sense severe infection via platelet TLR4 and then bind to and activate neutrophils to form NETs. Platelet-induced NET formation occurs very rapidly (in minutes) and may or may not result in death of the neutrophils. NETs formed in blood vessels can catch circulating bacteria as they pass through the vessels. Trapping of bacteria under flow has been imaged directly in flow chambers in vitro and intravital microscopy demonstrated that bacterial trapping occurs in the liver sinusoids and lung capillaries (sites where platelets bind neutrophils).

NET activation and release, or NETosis, is a dynamic process that can come in two forms, suicidal and vital NETosis. Overall, many of the key components of the process are similar for both types of NETosis, however, there are key differences in stimuli, timing, and ultimate end result. The full NETosis activation pathway is still under investigation but a few key proteins have been identified and slowly a full picture of the pathway is emerging. The process is thought to begin with NADPH oxidase activation of protein-arginine deiminase 4 (PAD4) via reactive-oxygen species (ROS) intermediaries. PAD4 is responsible for the citrullination of histones in the neutrophil, resulting in decondensation of chromatin. Azurophilic granule proteins such as myeloperoxidase (MPO) and neutrophil elastase (NE) then enter the nucleus and further the decondensation process, resulting in the rupture of the nuclear envelope. The uncondensed chromatin enter the cytoplasm where additional granule and cytoplasmic proteins are added to the early-stage NET. The end result of the process then depends on which NETosis pathway is activated.

Suicidal NETosis. Suicidal NETosis was first described in a 2007 study that noted that the release of NETs resulted in neutrophil-death through a different pathway than apoptosis or necrosis. In suicidal NETosis, the intracellular NET formation is followed by the rupture of the plasma membrane, releasing it into the extracellular space. This NETosis pathway can be initiated through activation of Toll-like Receptors (TLRs), Fc receptors, and complement receptors with various ligands such as antibodies, PMA, and so on. The current understanding is that upon activation of these receptors, downstream signaling results in the release of calcium from the endoplasmic reticulum. This intracellular influx of calcium in turn activates NADPH oxidase, resulting in activation of the NETosis pathway as described above. Of note, suicidal NETosis can take hours, even with high levels of PMA stimulation, while vital NETosis that can be completed in a matter of minutes.

Vital NETosis. Vital NETosis can be stimulated by bacterial lipopolysaccharide (LPS), other “bacterial products, TLR4-activated platelets, or complement proteins in tandem with TLR2 ligands.” Vital NETosis is made possible through the blebbing of the nucleus, resulting in a DNA-filled vesicle that is exocytosed and leaves the plasma membrane intact. Its rapid formation and release does not result in neutrophil death, however, the cell is without DNA, raising questions about whether a cell without DNA can be considered alive. It has been noted that neutrophils can continue to phagocytose and kill microbes after vital NETosis, highlighting the neutrophil's anti-microbial versatility.

Regulation.

The formation of NETs is regulated by the lipoxygenase pathway—during certain forms of activation (including contact with bacteria) neutrophil 5-lipoxygenase forms 5-HETE-phospholipids that inhibit NET formation. Evidence from laboratory experiments suggests that NETs are cleaned away by macrophages that phagocytose and degrade them.

NET-Associated Host Damage.

NETs might also have a deleterious effect on the host, because the exposure of extracellular histone complexes could play a role during the development of autoimmune diseases like systemic lupus erythematosus. NETs could also play a role in inflammatory diseases, as NETs could be identified in preeclampsia, a pregnancy related inflammatory disorder in which neutrophils are known to be activated. NETs have also been reported in the colon mucosa of patients with the inflammatory bowel disease ulcerative colitis. NETs have also been associated with the production of IgG antinuclear double stranded DNA antibodies in children infected with P. falciparum malaria.

NETs also have a role in thrombosis. These observations suggest that NETs might play an important role in the pathogenesis of infectious, inflammatory and thrombotic disorders.

### III. TRAIL, TRAIL FUSIONS AND CELLS EXPRESSING THE SAME

A. TRAIL

TNF-related apoptosis-inducing ligand (TRAIL), is a protein functioning as a ligand that induces the process of cell death called apoptosis. TRAIL is a cytokine that is produced and secreted by most normal tissue cells. It causes apoptosis primarily in tumor cells, by binding to certain death receptors. TRAIL and its receptors have been used as the targets of several anti-cancer therapeutics since the mid-1990s, such as Mapatumumab. However, as of 2013, these have not shown significant survival benefit. TRAIL has also been designated CD253 (cluster of differentiation 253) and TNF SF10 (tumor necrosis factor (ligand) superfamily, member 10).

In humans, the gene that encodes TRAIL is located at chromosome 3q26, which is not close to other TNF family members. The genomic structure of the TRAIL gene spans approximately 20 kb and is composed of five exonic segments 222, 138, 42, 106, and 1245 nucleotides and four introns of approximately 8.2, 3.2, 2.3 and 2.3 kB.

The TRAIL gene lacks TATA and CAAT boxes and the promotor region contains putative response elements for transcription factors GATA, AP-1, C/EBP, SP-1, OCT-1, AP3, PEA3, CF-1, and ISRE.

TIC10 (which causes expression of TRAIL) was investigated in mice with various tumor types. Small molecule ONC201 causes expression of TRAIL which kills some cancer cells.

TRAIL shows homology to other members of the tumor necrosis factor superfamily. It is composed of 281 amino acids and has characteristics of a type II transmembrane protein (i.e., no leader sequence and an internal transmembrane domain). The N-terminal cytoplasmic domain is not conserved across family members, however, the C-terminal extracellular domain is conserved and can be proteolytically cleaved from the cell surface. TRAIL forms a homotrimer that binds three receptor molecules.

TRAIL binds to the death receptors DR4 (TRAIL-RI) and DRS (TRAIL-MI). The process of apoptosis is caspase-8-dependent. Caspase-8 activates downstream effector caspases including procaspase-3, -6, and -7, leading to activation of specific kinases. TRAIL also binds the receptors DcR1 and DcR2, which do not contain a cytoplasmic domain (DcR1) or contain a truncated death domain (DcR2). DcR1 functions as a TRAIL-neutralizing decoy-receptor. The cytoplasmic domain of DcR2 is functional and activates NF-kappaB. In cells expressing DcR2, TRAIL binding therefore activates NF-kappaB, leading to transcription of genes known to antagonize the death signaling pathway and/or to promote inflammation. TRAIL has been shown to interact with TNFRSF10B.

In clinical trials only a small proportion of patients responded to various drugs that targeted TRAIL death receptors.

B. TRAIL Fusions

The present disclosure contemplates the construction and expression of TRAIL fusion proteins. In general, a fusion protein comprises two domains from distinct proteins that have been genetically “fused” by combining two coding regions in a 5′ to 3′ orientation, thereby permitting expression of a single protein product containing those two domains.

In the present disclosure, the fusion “partner” to be connected to TRAIL is, at a general level, a positively-charged polypeptide.

The surface charge of a protein of interest can be theoretically determined by summing up the number of positively and negatively charged amino acids (e.g. Lys, Arg, Asp, Glu) of the protein. The surface charge can also be empirically quantified using capillary electrophoresis and protein charge ladders.

The extracellular domain of TRAIL (starting with amino acid 94) containing the active site is sufficient to elicit the desired TRAIL-induced apoptotic effect. Any conjugating peptide onto TRAIL should be linked at the N-terminal of TRAIL to deter steric hindrance to TRAIL's active site which is located at the C-terminal. As for the positively charged protein itself, the orientation of the protein is less critical. For example, GFP is a highly stable protein with no distinct “active site” region. And thus it is not important where the conjugating end is. However, if the positively charged protein also has an active site and its ability to bind to ligands is required then it is highly advisable to design the conjugation site that would minimize any potential steric hindrance.

An exemplary protein is green fluorescent protein, or GFP. The green fluorescent protein (GFP) is a protein composed of 238 amino acid residues (26.9 kDa) that exhibits bright green fluorescence when exposed to light in the blue to ultraviolet range. Although many other marine organisms have similar green fluorescent proteins, GFP traditionally refers to the protein first isolated from the jellyfish Aequorea victoria. The GFP from A. victoria has a major excitation peak at a wavelength of 395 nm and a minor one at 475 nm. Its emission peak is at 509 nm, which is in the lower green portion of the visible spectrum. The fluorescence quantum yield (QY) of GFP is 0.79. The GFP from the sea pansy (Renilla reniformis) has a single major excitation peak at 498 nm. GFP makes for an excellent tool in many forms of biology due to its ability to form internal chromophore without requiring any accessory cofactors, gene products, or enzymes/substrates other than molecular oxygen.

The GFP gene is frequently used as a reporter of expression. It has been used in modified forms to make biosensors, and many animals have been created that express GFP, which demonstrates a proof of concept that a gene can be expressed throughout a given organism, in selected organs, or in cells of interest. GFP can be introduced into animals or other species through transgenic techniques, and maintained in their genome and that of their offspring. To date, GFP has been expressed in many species, including bacteria, yeasts, fungi, fish and mammals, including in human cells.

In the 1960s and 1970s, GFP, along with the separate luminescent protein aequorin (an enzyme that catalyzes the breakdown of luciferin, releasing light), was first purified from Aequorea victoria and its properties studied. In A. victoria, GFP fluorescence occurs when aequorin interacts with Ca′ ions, inducing a blue glow. Some of this luminescent energy is transferred to the GFP, shifting the overall color towards green. However, its utility as a tool for molecular biologists did not begin to be realized until 1992 when the cloning and nucleotide sequence of wtGFP was reported. Others then expressed the coding sequence of wtGFP, with the first few amino acids deleted, in heterologous cells of E. coli and C. elegans. Remarkably, the GFP molecule folded and was fluorescent at room temperature, without the need for exogenous cofactors specific to the jellyfish. Although this near-wtGFP was fluorescent, it had several drawbacks, including dual peaked excitation spectra, pH sensitivity, chloride sensitivity, poor fluorescence quantum yield, poor photostability and poor folding at 37° C.

The first reported crystal structure of a GFP was that of the S65T mutant in 1996. Shortly thereafter, the wild-type GFP structure was reported. These crystal structures provided vital background on chromophore formation and neighboring residue interactions. Researchers have modified these residues by directed and random mutagenesis to produce the wide variety of GFP derivatives in use today.

Due to the potential for widespread usage and the evolving needs of researchers, many different mutants of GFP have been engineered. The first major improvement was a single point mutation (S65T) reported in 1995. This mutation dramatically improved the spectral characteristics of GFP, resulting in increased fluorescence, photostability, and a shift of the major excitation peak to 488 nm, with the peak emission kept at 509 nm. This matched the spectral characteristics of commonly available FITC filter sets, increasing the practicality of use by the general researcher. A 37° C. folding efficiency (F64L) point mutant to this scaffold, yielding enhanced GFP (EGFP), was discovered in 1995. EGFP allowed the practical use of GFPs in mammalian cells. EGFP has an extinction coefficient (denoted ε) of 55,000 M⁻¹ cm⁻¹. The fluorescence quantum yield (QY) of EGFP is 0.60. The relative brightness, expressed as ε—QY, is 33,000 M⁻¹ cm⁻¹. Superfolder GFP, a series of mutations that allow GFP to rapidly fold and mature even when fused to poorly folding peptides, was reported in 2006.

Many other mutations have been made, including color mutants; in particular, blue fluorescent protein (EBFP, EBFP2, Azurite, mKalamal), cyan fluorescent protein (ECFP, Cerulean, CyPet, mTurquoise2), and yellow fluorescent protein derivatives (YFP, Citrine, Venus, YPet). BFP derivatives (except mKalamal) contain the Y66H substitution. They exhibit a broad absorption band in the ultraviolet centered close to 380 nanometers and an emission maximum at 448 nanometers. A green fluorescent protein mutant (BFPms1) that preferentially binds Zn(II) and Cu(II) has been developed. BFPms1 have several important mutations including and the BFP chromophore (Y66H), Y145F for higher quantum yield, H148G for creating a hole into the beta-barrel and several other mutations that increase solubility. Zn(II) binding increases fluorescence intensity, while Cu(II) binding quenches fluorescence and shifts the absorbance maximum from 379 to 444 nm. Therefore, they can be used as Zn biosensor.

The critical mutation in cyan derivatives is the Y66W substitution, which causes the chromophore to form with an indole rather than phenol component. Several additional compensatory mutations in the surrounding barrel are required to restore brightness to this modified chromophore due to the increased bulk of the indole group. In ECFP and Cerulean, the N-terminal half of the seventh strand exhibits two conformations. These conformations both have a complex set of van der Waals interactions with the chromophore. The Y145A and H148D mutations in Cerulean stabilize these interactions and allow the chromophore to be more planar, better packed, and less prone to collisional quenching. Additional site-directed random mutagenesis in combination with fluorescence lifetime based screening has further stabilized the seventh β-strand resulting in a bright variant, mTurquoise2, with a quantum yield (QY) of 0.93. The red-shifted wavelength of the YFP derivatives is accomplished by the T203Y mutation and is due to π-electron stacking interactions between the substituted tyrosine residue and the chromophore. These two classes of spectral variants are often employed for Förster resonance energy transfer (FRET) experiments. Genetically encoded FRET reporters sensitive to cell signaling molecules, such as calcium or glutamate, protein phosphorylation state, protein complementation, receptor dimerization, and other processes provide highly specific optical readouts of cell activity in real time.

Semirational mutagenesis of a number of residues led to pH-sensitive mutants known as pHluorins, and later super-ecliptic pHluorins. By exploiting the rapid change in pH upon synaptic vesicle fusion, pHluorins tagged to synaptobrevin have been used to visualize synaptic activity in neurons. Redox sensitive versions of GFP (roGFP) were engineered by introduction of cysteines into the beta barrel structure. The redox state of the cysteines determines the fluorescent properties of roGFP.

The nomenclature of modified GFPs is often confusing due to overlapping mapping of several GFP versions onto a single name. For example, mGFP often refers to a GFP with an N-terminal palmitoylation that causes the GFP to bind to cell membranes. However, the same term is also used to refer to monomeric GFP, which is often achieved by the dimer interface breaking A206K mutation. Wild-type GFP has a weak dimerization tendency at concentrations above 5 mg/mL. mGFP also stands for “modified GFP,” which has been optimized through amino acid exchange for stable expression in plant cells.

In addition to supercharged eGFP-linked TRAIL to facilitate NET binding, the following naturally existing proteins are also potential targets for TRAIL conjugation due to their presence in NETs: MPO, ELA-2, Proteinase 3, Lactotransferin, Caspesin G (Urban et al., 2009).

C. Other Proapoptotic Molecules

As an alternative to the TRAIL fusions described above, one may also employ other proapoptotic molecules. For example, Fas ligand (FasL or CD95L) is a type-II transmembrane protein that belongs to the tumor necrosis factor (TNF) family, and its binding with its receptor induces apoptosis. Fas ligand/receptor interactions play an important role in the regulation of the immune system and the progression of cancer. Fas ligand or FasL is a homotrimeric type II transmembrane protein expressed on cytotoxic T lymphocytes. It signals through trimerization of FasR, which spans the membrane of the “target” cell. This trimerization usually leads to apoptosis, or cell death. Soluble Fas ligand is generated by cleaving membrane-bound FasL at a conserved cleavage site by the external matrix metalloproteinase MMP-7.
FasR: The Fas receptor (FasR), or CD95, is the most intensely studied member of the death receptor family. The gene is situated on chromosome 10 in humans and 19 in mice. Previous reports have identified as many as eight splice variants, which are translated into seven isoforms of the protein. Many of these isoforms are rare haplotypes that are usually associated with a state of disease. Apoptosis-inducing Fas receptor is dubbed isoform 1 and is a type 1 transmembrane protein. It consists of three cysteine-rich pseudorepeats, a transmembrane domain, and an intracellular death domain.
DcR3: Decoy receptor 3 (DcR3) is a recently discovered decoy receptor of the tumor necrosis factor superfamily that binds to FasL, LIGHT, and TL1A. DcR3 is a soluble receptor that has no signal transduction capabilities (hence a “decoy”) and functions to prevent FasR-FasL interactions by competitively binding to membrane-bound Fas ligand and rendering them inactive.

Apoptosis triggered by Fas-Fas ligand binding plays a fundamental role in the regulation of the immune system. Its functions include:
T-cell homeostasis: the activation of T-cells leads to their expression of the Fas ligand. T cells are initially resistant to Fas-mediated apoptosis during clonal expansion, but become progressively more sensitive the longer they are activated, ultimately resulting in activation-induced cell death (AICD). This process is needed to prevent an excessive immune response and eliminate autoreactive T-cells. Humans and mice with deleterious mutations of Fas or Fas ligand develop an accumulation of aberrant T-cells, leading to lymphadenopathy, splenomegaly, and lupus erythematosus.
Cytotoxic T-cell activity: Fas-induced apoptosis and the perforin pathway are the two main mechanisms by which cytotoxic T lymphocytes induce cell death in cells expressing foreign antigens.
Immune privilege: Cells in immune privileged areas such as the cornea or testes express Fas ligand and induce the apoptosis of infiltrating lymphocytes. It is one of many mechanisms the body employs in the establishment and maintenance of immune privilege.
Maternal tolerance: Fas ligand may be instrumental in the prevention of leukocyte trafficking between the mother and the fetus, although no pregnancy defects have yet been attributed to a faulty Fas-Fas ligand system.
Tumor counterattack: Tumors may over-express Fas ligand and induce the apoptosis of infiltrating lymphocytes, allowing the tumor to escape the effects of an immune response. The up-regulation of Fas ligand often occurs following chemotherapy, from which the tumor cells have attained apoptosis resistance.

Another proapoptotic molecule is p28. This 28-residue peptide, derived from azurin, a redox protein secreted by P. aeruginosa, produces a post-translation increase in the level in p53 and inhibits the cell cycle at G₂/M. Arginine deaminase (ADI) is a microbial enzyme from Mycoplasma produced in E. coli. It has high affinity to L-arginine and hydrolyzes L-arginine to citrulline and ammonia. Low concentrations of ADI have been shown to inhibit proliferation in certain cultured cells by arresting the cell cycle in G₁ and/or S phase.

D. Neutrophil Transformation and Expression

In certain embodiments, neutrophils will be engineered to express TRAIL fusion proteins. This can be accomplished by transfecting mRNA encoding the fusion proteins or transforming the neutrophils with expression cassettes encoding the fusion protein. The neutrophils, once engineered for expression, can be introduced (or re-introduced in the case of autologous methods) to a subject. In either case, effective expression requires that appropriate signals be provided in the nucleic acids and vectors, including various regulatory elements such as enhancers/promoters from both viral and mammalian sources that drive expression of the fusion proteins. Elements designed to optimize messenger RNA stability and translatability in host cells also may be employed. The conditions for the use of a number of dominant drug selection markers for establishing stable and selected expression are contemplated.

Note that neutrophils have a limited lifespan in the peripheral blood, of approximately 24 hours or less, and so an effective approach to engineering neutrophils is to transfect pluripotent hematopoietic stem cells with the TRAIL construct together with a neutrophil-specific promoter that would only express the supercharged trail in differentiated hemotopoietic cells of the neutrophil/granulocyte lineage. The inventors previously used such an approach to induce expression of transmembrane TRAIL only in the megakaryocyte/platelet lineage (Li et al., 2016), although the prior work did not involve any of the novel netosis and supercharged TRAIL aspects described here.

1. Regulatory Elements

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct (Table 2 and Table 3). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin | Banerji et al., 1983; Gilles et al., 1983; |
| Heavy Chain | Grosschedl et al., 1985; Atchinson et al., |
| | 1986, 1987; Imler et al., 1987; Weinberger |
| | et al., 1984; Kiledjian et al., 1988; Porton |
| | et al.; 1990 |
| Immunoglobulin | Queen et al., 1983; Picard et al., 1984 |
| Light Chain | |
| T-Cell Receptor | Luria et al., 1987; Winoto et al., 1989; |
| | Redondo et al.; 1990 |
| HLA DQ a and/or | Sullivan et al., 1987 |
| DQ β | |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA- | Sherman et al., 1989 |
| DRa | |
| β-Actin | Kawamoto et al., 1988; Ng et al.; 1989 |
| Muscle Creatine | Jaynes et al., 1988; Horlick et al., 1989; |
| Kinase (MCK) | Johnson et al., 1989 |
| Prealbumin | Costa et al., 1988 |
| (Transthyretin) | |
| Elastase I | Ornitz et al., 1987 |
| Metallothionein | Karin et al., 1987; Culotta et al., 1989 |
| (MTII) | |
| Collagenase | Pinkert et al., 1987; Angel et al., 1987a |
| Albumin | Pinkert et al., 1987; Tronche et al., 1989, |
| | 1990 |
| α-Fetoprotein | Godbout et al., 1988; Campere et al., 1989 |
| t-Globin | Bodine et al., 1987; Perez-Stable et al., |
| | 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion | Hirsh et al., 1990 |
| Molecule (NCAM) | |
| α₁-Antitrypain | Latimer et al., 1990 |
| H2B (TH2B) Histone | Hwang et al., 1990 |
| Mouse and/or Type I | Ripe et al., 1989 |
| Collagen | |
| Glucose-Regulated | Chang et al., 1989 |
| Proteins (GRP94 and | |
| GRP78) | |
| Rat Growth Hormone | Larsen et al., 1986 |
| Human Serum | Edbrooke et al., 1989 |
| Amyloid A (SAA) | |
| Troponin I (TN I) | Yutzey et al., 1989 |
| Platelet-Derived | Pech et al., 1989 |
| Growth Factor | |
| (PDGF) | |
| Duchenne Muscular | Klamut et al., 1990 |
| Dystrophy | |
| SV40 | Banerji et al., 1981; Moreau et al., 1981; |
| | Sleigh et al., 1985; Firak et al., 1986; Herr |
| | et al., 1986; Imbra et al., 1986; Kadesch |
| | et al., 1986; Wang et al., 1986; Ondek |
| | et al., 1987; Kuhl et al., 1987; Schaffner |
| | et al., 1988 |
| Polyoma | Swartzendruber et al., 1975; Vasseur et al., |
| | 1980; Katinka et al., 1980, 1981; Tyndell |
| | et al., 1981; Dandolo et al., 1983; de |
| | Villiers et al., 1984; Hen et al., 1986; |
| | Satake et al., 1988; Campbell and |
| | Villarreal, 1988 |
| Retroviruses | Kriegler et al., 1982, 1983; Levinson et al., |
| | 1982; Kriegler et al., 1983, 1984a, b, 1988; |
| | Bosze et al., 1986; Miksicek et al., 1986; |
| | Celander et al., 1987; Thiesen et al., 1988; |
| | Celander et al., 1988; Choi et al., 1988; |
| | Reisman et al., 1989 |
| Papilloma Virus | Campo et al., 1983; Lusky et al., 1983; |
| | Spandidos and/or Wilkie, 1983; Spalholz |
| | et al., 1985; Lusky et al., 1986; Cripe |
| | et al., 1987; Gloss et al., 1987; Hirochika |
| | et al., 1987; Stephens et al., 1987 |
| Hepatitis B Virus | Bulla et al., 1986; Jameel et al., 1986; |
| | Shaul et al., 1987; Spandau et al., 1988; |
| | Vannice et al., 1988 |
| Human Immuno- | Muesing et al., 1987; Hauber et al., 1988; |
| deficiency Virus | Jakobovits et al., 1988; Feng et al., 1988; |
| | Takebe et al., 1988; Rosen et al., 1988; |
| | Berkhout et al., 1989; Laspia et al., 1989; |
| | Sharp et al., 1989; Braddock et al., 1989 |
| Cytomegalovirus | Weber et al., 1984; Boshart et al., 1985; |
| (CMV) | Foecking et al., 1986 |
| Gibbon Ape | Holbrook et al., 1987; Quinn et al., 1989 |
| Leukemia Virus | |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1981; Lee |
| mammary tumor | | et al., 1981; Majors et al., |
| virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene | Interferon | Blanar et al., 1989 |
| H-2κb | | |
| HSP70 | ElA, SV40 Large T | Taylor et al., 1989, |
| | Antigen | 1990a, 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Other neutrophil-specific promoters include myeloid related protein 8 (MRP-8) promoter, myeloid related protein 14 (MRP-14) promoter, neutrophil elase promoter, CD11b promoter, and lysozyme C promoter.

Where a cDNA insert is employed, one will typically desire to include a polyadenylation signal to effect proper polyadenylation of the gene transcript. The nature of the polyadenylation signal is not believed to be crucial to the successful practice of the disclosure, and any such sequence may be employed such as human growth hormone and SV40 polyadenylation signals. Also contemplated as an element of the expression cassette is a terminator. These elements can serve to enhance message levels and to minimize read through from the cassette into other sequences.

B. Delivery of Nucleic Acids and Expression Vectors

Non-viral methods for the transfer of mRNAs and expression constructs into cells also are contemplated by the present disclosure. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

The nucleic acid expression construct may simply consist of naked mRNA, DNA or plasmid. Transfer of such constructs may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In another embodiment, the disclosure relates to transferring a naked nucleic acid into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

In a further embodiment, the nucleic acid may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes. A reagent known as Lipofectamine 2000™ is widely used and commercially available.

Another approach to cell transformation is through the use of a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One method for delivery of an expression construct to a neutrophil involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNAs issued from this promoter possess a 5′-tripartite leader (TPL) sequence which makes them preferred mRNAs for translation.

Another class of viral vectors—the retroviruses—are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Other viral vectors may be employed as expression constructs in the present disclosure. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

### IV. TREATMENT OF CANCERS

A. Cancers

In some embodiments, the present disclosure provides methods for treating cancer. These methods are designed to treat or delay progression of cancer in an individual comprising administering to the individual an effective amount a neutrophil engineered to express a TRAIL fusion protein.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; hodgkin's; paragranuloma; malignant lymphoma, small lymphocytic; malignant lymphoma, large cell, diffuse; malignant lymphoma, follicular; mycosis fungoides; other specified non-Hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); chronic myeloblastic leukemia (CIVIL); and blastic plasmacytoid dendritic cell neoplasm (BPDCN).

In some cases, the individual is provided with one or more doses of the engineered neutrophils. In cases where the individual is provided with two or more doses, the duration between the administrations may be 1, 2, 3, 4, 5, 6, 7, or more days. Therapeutically effective amounts of neutrophils can be administered by a number of routes, including parenteral administration, for example, intravenous, intraperitoneal, intramuscular, intrasternal, or intraarticular injection, or infusion.

The engineered neutrophils can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective number of engineered neutrophils will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ engineered neutrophils/m². In a certain embodiment, the dose used in the treatment of human subjects ranges from about 3.8×10⁹ to about 3.8×10¹⁰ engineered neutrophils/m². In additional embodiments, a therapeutically effective number of engineered neutrophils can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact number of engineered neutrophils is readily determined by one of skill in the art based on the age, weight, sex, and physiological condition of the subject. Effective doses can be extrapolated from dose-response curves derived from in vitro or animal model test systems.

B. Pharmaceutical Compositions

Also provided herein are pharmaceutical compositions and formulations comprising engineered neutrophils and a pharmaceutically acceptable carrier. Pharmaceutical compositions and formulations as described herein can be prepared by mixing the active ingredients (i.e., an engineered neutrophil) having the desired degree of purity with one or more optional pharmaceutically acceptable carriers (Remington's Pharmaceutical Sciences 22^{nd} edition, 2012), in the form of aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

C. Combination Therapies

In certain embodiments, the compositions and methods of the present embodiments involve an engineered neutrophil population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

A neutrophil therapy may be administered before, during, after, or in various combinations relative to an additional cancer therapy, such as immune checkpoint therapy. The administrations may be in intervals ranging from concurrently to minutes to days to weeks. In some embodiments where the engineered neutrophil therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an engineered neutrophil therapy is “A” and an anti-cancer therapy is “B”:

| | | | | | |
|---|---|---|---|---|---|
| A/B/A | B/A/B | B/B/A | A/A/B | A/B/B | B/A/A |
| A/B/B/B | B/A/B/B | B/B/B/A | B/B/A/B | A/A/B/B | A/B/A/B |
| A/B/B/A | B/B/A/A | B/A/B/A | B/A/A/B | A/A/A/B | B/A/A/A |
| A/B/A/A | A/A/B/A | | | | |

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

1. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammalI and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarb azine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

2. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

3. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells.

Antibody-drug conjugates have emerged as a breakthrough approach to the development of cancer therapeutics. Cancer is one of the leading causes of deaths in the world. Antibody—drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD20, carcinoembryonic antigen, tyrosinase (p9′7), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, γ-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 axis antagonists for use in the methods provided herein are known in the art such as described in U.S. Patent Publication Nos. 20140294898, 2014022021, and 20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### V. EXAMPLES

The following examples are included to demonstrate preferred embodiments. It should be appreciated by those of skill in the art that the techniques disclosed in the examples that follow represent techniques discovered by the inventors to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### A. Materials and Methods

Neutrophils isolated from human donors were electroporated with mRNA sequence encoding human TRAIL N-terminally linked with a mutated form of eGFP protein that has a highly positive surface charge. After 24 hr, PMA or IL-8 was added to transfected neutrophils to induce netosis. Fluorescent imaging was taken at indicated time points to visualize the localization of GFP-TRAIL protein onto NETs. Annexin V/PI staining was performed to assess apoptosis of cancer cell co-cultures with transfected neutrophils.

### B. Results and Discussion

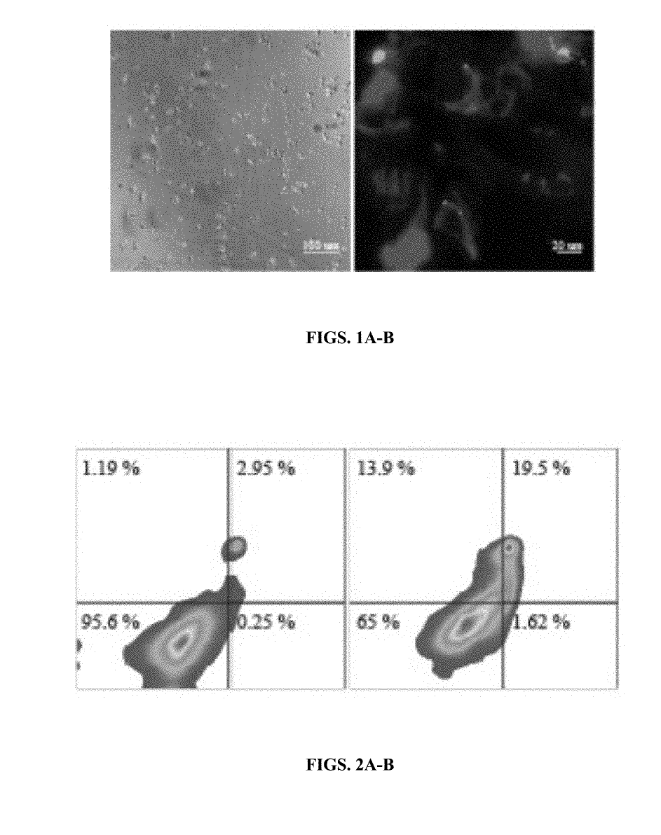

mRNA sequence of positively-charged GFP-TRAIL was successfully transfected into human neutrophils. As early as 4 hours, neutrophils began expressing GFP-TRAIL protein (FIG. 1A). Most transfected neutrophils retained normal morphology and neutrophilic functions as evident by their response to netosis stimuli. Importantly, NETs released by transfected neutrophils were decorated with GFP-TRAIL (FIG. 1B). To test the apoptosis potency of TRAIL-decorated NETs, the inventor co-cultured multiple cancer cell lines with the transfected neutrophils overnight. Apoptosis assay was then performed on the cancer cells and a significant increase in apoptosis signal was observed, both early and late stages of cell death as indicated by FIG. 2A (control) and FIG. 2B.

In this study, the inventor successfully re-engineered human neutrophils capable of expressing TRAIL-decorated NETs when undergoing netosis in the presence of cancer cells. Importantly, TRAIL-decorated NETs are capable ensnaring cancer cells and induce cell death and represent a potential new approach to cancer treatment.

### Example 2

### A. Materials and Methods

Cell Culture and Differentiation.

The acute myeloid leukemia PLB-985 (derivative of HL-60) cell line stably expressing Cas9 protein was a generous gift from the Collins Lab (University of California, Davis). The cells were cultured in RPMI 1640 media supplemented with 2 mM L-glutamine, 25 mM HEPES, 10% (v/v) FBS, and 2 μg/mL Blastincidin at 37° C. and 5% CO₂. Cultured cells were regularly test for mycoplasma using Universal Mycoplasma Detection Kit (ATCC 30-1012K).

Neutrophil differentiation of logarithmically growing PLB-985 cells was induced by reduction of FBS to 5% and supplementation of 0.5% (vol/vol) DMF. After 3 days, an equivalent of the initial volume of differentiating medium was added and the differentiation continued until day 7.

CRISPR/CAS9 Knock-in of eGFP-TRAIL.

2×10⁶ PLB-985 cells were nucleofected (AMAXA Cell Nucleofector Kit V and Amaxa Nucleofactor II, program C-023) With 20 μg of pAAVS1-eGFP-TRAIL and 20 μg of pX330-U6-AAVS1. Immediately after nucleofection 500 μl of medium was added to the cuvette and the cells were incubated at room temp. for 10 minutes. Then cell were cultured for 48 hours. After 48 hours, GFP expressing cells were selected with culture medium supplemented with 2 μg/mL Puromycin.

Chemical and Antibodies.

TACS® Annexin V Kit (Gaithersburg, Md., USA) was used for assaying cell apoptosis. Reagents for SEM were obtained from Electron Microscopy Sciences (Hatfield, Pa., USA): glutaraldehyde, osmium tetroxide and uranyl acetate. Antibodies for human TRAIL were purchased from Biolegend (San Diego, Calif., USA). Primary antibodies for human TRAIL and (3-actin were obtained from PeproTech (Rocky Hill, N.J., USA) and Santa Cruz Biotech (Santa Cruz, Calif., USA). HRP-conjugated anti-mouse and anti-rabbit antibodies were from Santa Cruz Biotech. IL-8 human Elisa Kits were purchased from ThermoFisher.

### B. Results

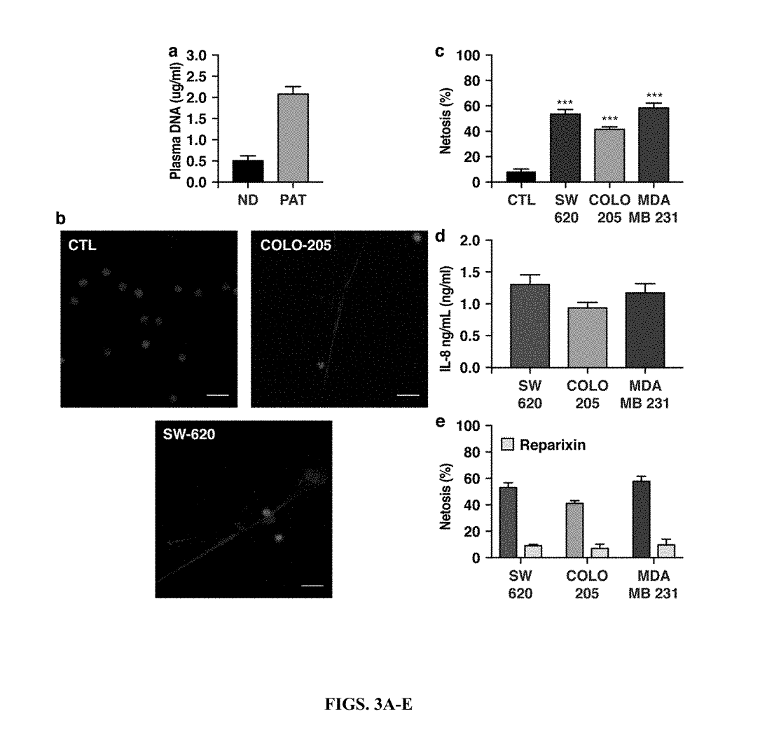

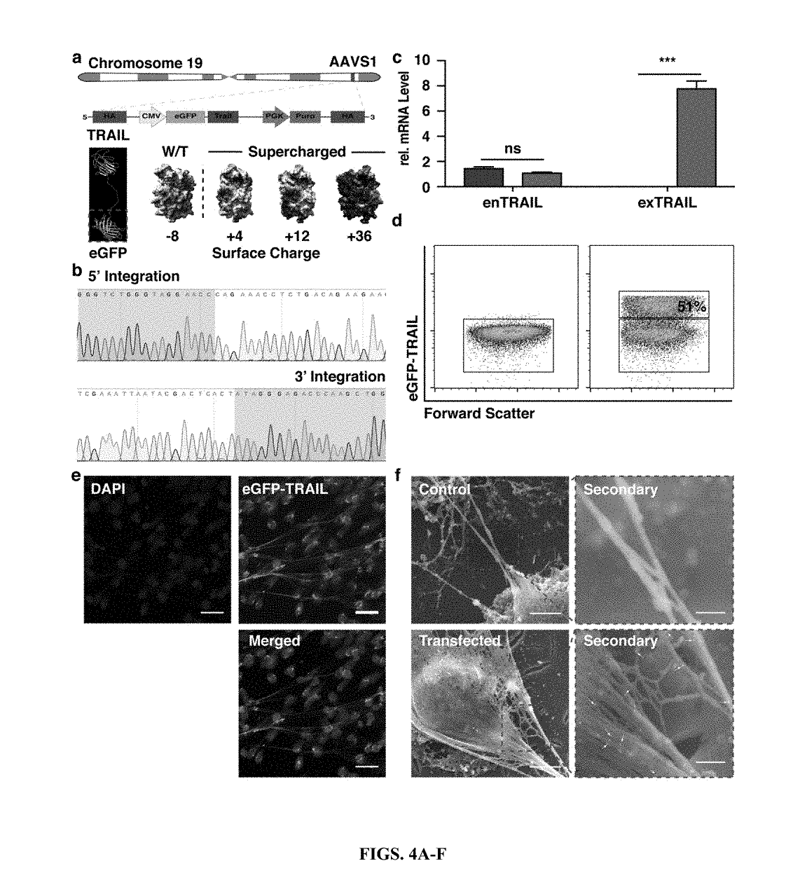

NETosis is a unique form of innate immune response elicited primarily by neutrophils to combat microbial infections (Brinkmann et al., 2004). In the presence of antigens, neutrophils undergo NETosis by following a program of cell death and releasing condensed DNA fibers decorated with cationic antimicrobial proteins, collectively called NETs, into the extracellular space (Papayannopoulous, 2018). Tumor cells release tumor-derived interleukin 8 (IL-8), a potent neutrophil chemoattractant, to increase tumor growth and metastatic potential by (a) promote tumor neovascularization and (b) induce infiltrating neutrophils to release pro-metastatic enzymes (De Larco et al., 2004). Initially, NETosis was found to specifically occur in the presence of bacterial antigens; however, the inventors show here that tumor-derived IL-8 released by tumor cells can also elicit NETosis in neutrophils (FIGS. 3B-C). Blocking the tumor-derived IL-8 pathway with a small molecule, reparixin, returned NETosis back to basal level (FIG. 3E). Moreover, the inventors quantified cell-free serum DNA levels in cancer patients and found a significant increase in serum DNA level compared to the healthy cohort suggesting that NETs may also have an elevated presence in the circulatory system in cancer patients (FIG. 3A). These results support previous findings that malignant and non-malignant neutrophils in tumor-bearing mice have increased sensitivity toward undergoing NETosis (Demers et al., 2012). Given data shown here of the ubiquitous presence of neutrophils in all stages of tumor development, the inventors propose a unique anti-tumor drug delivery system by exploiting the ability of neutrophils to spontaneously undergo NETosis in the presence of cancer cells.

Although NETs have been shown to ensnare cancer cells, they lack the ability to kill cancer cells (data not shown). More insidiously, trapped tumor cells show increased metastatic potential (Giannias et al., 2013). In the current study, the inventors proposed to re-engineer human neutrophils to express NETs decorated with an apoptosis-inducing peptide, TRAIL, that could selectively destroy cancer cells during NETosis. TRAIL is a small cytokine expressed by most cell types that selectively induces apoptosis in tumor cells overexpressing death receptors while sparing healthy cells (Mitchell et al., 2014; Li et al., 2016). In this model, the inventors successfully knocked in the gene of interest (GOI), expressing the chimeric protein eGFP-TRAIL, into the safe harbor site AAVS1 on chromosome 19 of a proto-neutrophilic cell line PLB-985 using the CRISPR/Cas system (FIGS. 4A-B). PLB-985 is leukemic cell line that and be induced to neutrophil-like cells capable of undergoing NETosis (Pedruzzi et al., 2002; Lievin-Le Moal et al., 2012). Aside from serving as a fluorescent marker, eGFP serves a more important function by selectively allowing the chimeric protein to electrostatically bind to the DNA fibers of NETs during NETosis (FIG. 4A). By modifying the surface charge of eGFP to become increasingly more positive, the inventors were able to increase its avidity to the negatively-charged DNA fibers in a charge-dependent manner (data not shown). The transfected cells stably expressed eGFP-TRAIL-decorated NETs during NETosis (FIGS. 4E-F). mRNA data showed successful expression of the transgene (FIGS. 4C-D). Immunostaining scanning electron microscopy with TRAIL antibody-conjugated gold nanoparticles of NET DNA fibers revealed that eGFP-TRAIL protein molecules are decorated along the DNA fibers (FIG. 4F).

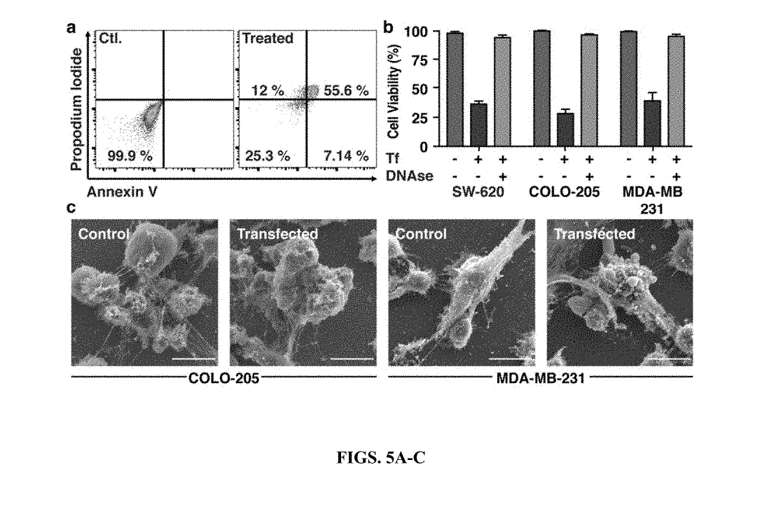

PLB-985 derived neutrophils expressing NETs decorated with eGFP-TRAIL co-cultured with multiple human tumor cell lines showed significant apoptosis-inducing potential. In COLO-205, after 16 hours of co-culture, 7% and 12% of the tumor cells underwent early and late apoptosis, respectively, while over 55% of the population were classify as necrosis (FIG. 5A). In the presence of eGFP-decorated NETs the inventors observed an over 60% kill rate of cancer cells in the three cell lines SW620, COLO 205 and MDA-MB-213 (FIG. 5B). SEM images clearly showed significant NETosis when neutrophils were co-cultured with tumor cells. However, only when NETs were decorated with eGFP-TRAIL did the tumor cells undergo apoptosis as evident by membrane blebbing (FIG. 5C).

The constant presence of neutrophils near tumor cells in all stages of cancer development and in conjunction with the unique ability of neutrophil to undergo NETosis in the presence of cancer cells, make neutrophils a promising candidate for the delivery cancer therapeutics. Here the inventors introduced a novel form of cancer therapy by leveraging these unique aspects of neutrophils by re-engineering the cell to express TRAIL. This form of treatment exemplifies the potential of employing neutrophils as drug delivery vehicles that, until now, have been largely unexplored.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
Brinkmann et al., Science, 303, 1532.1535, 2004.
Hidalgo et al., J Immunol Res., 2015.
Cool-Lartique et al., J Clin Invest., 123(8), 3446-3458, 2013.
Lee et al., J. Biol. Chem. 282(7), 4812-4820, 2007.
Mitchell & King, Biophys J 102(8):, 1804-1814, 2012.
Urban et al., Plos Pathog., 5(10):e1000639, 2009.
Potter and Haley, Meth. Enzymol., 91, 613-633, 1983.
Li et al., J Controlled Release, 228:38-47, 2016.
Hurwitz et al. (1998) Proc Natl Acad Sci USA 95(17): 10067-10071.
Camacho et al. (2004) J Clin Oncology 22(145): Abstract No. 2505.
Mokyr et al. (1998) Cancer Res 58:5301-5304.
Pardoll, Nat Rev Cancer, 12(4): 252-64, 2012b.
Hui and Hashimoto, Infection Immun., 66(11):5329-5336, 1998.
Christodoulides et al., Microbiology, 144(Pt 11):3027-3037, 1998.
Bukowski et al., Clinical Cancer Res., 4(10):2337-2347, 1998.
Davidson et al., J Immunother 21(5):389-398, 1998.
Hellstrand et al., Acta Oncologica, 37(4):347-353, 1998.
Qin et al., Proc. Natl. Acad. Sci. USA, 95(24):14411-14416, 1998.
Austin-Ward and Villaseca, Revista Medica de Chile, 126(7):838-845, 1998.
Hollander, Front. Immun., 3:3, 2012.
Hanibuchi et al., Int. J Cancer, 78(4):480-485, 1998.
U.S. Patent Publication No. US20050260186
U.S. Patent Publication No. US20060104968
U.S. Patent Publication No. US20140294898
U.S. Patent Publication No. US20140022021
U.S. Patent Publication No. US20110008369
U.S. Pat. No. 5,824,311
U.S. Pat. No. 5,844,905
U.S. Pat. No. 5,885,796
U.S. Pat. No. 5,801,005
U.S. Pat. No. 5,739,169
U.S. Pat. No. 5,830,880
U.S. Pat. No. 5,846,945
U.S. Pat. No. 6,207,156
U.S. Pat. No. 8,735,553
U.S. Pat. No. 8,354,509
U.S. Pat. No. 8,008,449
U.S. Pat. No. 8,017,114
U.S. Pat. No. 8,119,129
U.S. Pat. No. 8,329,867
WO2009/114335
WO2009/101611
WO2010/027827
WO2011/066342
WO2015016718
WO2001014424
WO2000037504
WO1995001994
WO1998042752
Angel et al., Cell, 49:729, 1987b.
Angel et al., Mol. Cell. Biol., 7:2256, 1987a.
Baichwal and Sugden, In: Gene Transfer, Kucherlapati (Ed), NY, Plenum Press, 117-148, 1986.
Banerji et al., Cell, 27(2 Pt 1):299-308, 1981.
Banerji et al., Cell, 33(3):729-740, 1983.
Benvenisty and Neshif, Proc. Natl. Acad. Sci. USA, 83:9551-9555, 1986.
Berkhout et al., Cell, 59:273-282, 1989.
Blanar et al., EMBO 1, 8:1139, 1989.
Bodine and Ley, EMBO 1, 6:2997, 1987.
Boshart et al., Cell, 41:521, 1985.
Bosze et al., EMBO 5(7):1615-1623, 1986.
Braddock et al., Cell, 58:269, 1989.
Bulla and Siddiqui, J. Virol., 62:1437, 1986.
Campbell and Villarreal, Mol. Cell. Biol., 8:1993, 1988.
Campere and Tilghman, Genes and Dev., 3:537, 1989.
Campo et al., Nature, 303:77, 1983.
Celander and Haseltine, J. Virology, 61:269, 1987.
Celander et al., J. Virology, 62:1314, 1988.
Chandler et al., Cell, 33:489, 1983.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7:2745-2752, 1987.
Choi et al., Cell, 53:519, 1988.
Coffin, In: Virology, Fields et al. (Eds.), Raven Press, NY, 1437-1500, 1990.
Cohen et al., J. Cell. Physiol., 5:75, 1987.
Costa et al., Mol. Cell. Biol., 8:81, 1988.
Coupar et al., Gene, 68:1-10, 1988.
Cripe et al., EMBO 1, 6:3745, 1987.
Culotta and Hamer, Mol. Cell. Biol., 9:1376, 1989.
Dandolo et al., J. Virology, 47:55-64, 1983.
De Villiers et al., Nature, 312(5991):242-246, 1984.
Deschamps et al., Science, 230:1174-1177, 1985.
Dubensky et al., Proc. Natl. Acad. Sci. USA, 81:7529-7533, 1984.
Edbrooke et al., Mol. Cell. Biol., 9:1908, 1989.
Edlund et al., Science, 230:912-916, 1985.
Fechheimer et al., Proc Natl. Acad. Sci. USA, 84:8463-8467, 1987.
Feng and Holland, Nature, 334:6178, 1988.
Firak and Subramanian, Mol. Cell. Biol., 6:3667, 1986.
Foecking and Hofstetter, Gene, 45(1):101-105, 1986.
Fraley et al., Proc Natl. Acad. Sci. USA, 76:3348-3352, 1979
Friedmann, Science, 244:1275-1281, 1989.
Fujita et al., Cell, 49:357, 1987.
Ghosh and Bachhawat, In: Liver Diseases, Targeted Diagnosis and Therapy Using Specific Receptors and Ligands, Wu et al. (Eds.), Marcel Dekker, NY, 87-104, 1991.
Gilles et al., Cell, 33:717, 1983.
Gloss et al., EMBO J., 6:3735, 1987.
Godbout et al., Mol. Cell. Biol., 8:1169, 1988.
Goodbourn and Maniatis, Proc. Natl. Acad. Sci. USA, 85:1447, 1988.
Goodbourn et al., Cell, 45:601, 1986.
Gopal, Mol. Cell Biol., 5:1188-1190, 1985.
Graham and van der Eb, Virology, 52:456-467, 1973.
Greene et al., Immunology Today, 10:272, 1989
Grosschedl and Baltimore, Cell, 41:885, 1985.
Grunhaus and Horwitz, Seminar in Virology, 3:237-252, 1992.
Harland and Weintraub, J. Cell Biol., 101:1094-1099, 1985.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hirochika et al., J. Virol., 61:2599, 1987.
Holbrook et al., Virology, 157:211, 1987.
Horlick and Benfield, Mol. Cell. Biol., 9:2396, 1989.
Horwich et al., J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Jaynes et al., Mol. Cell. Biol., 8:62, 1988.
Johnson et al., Mol. Cell. Biol., 9:3393, 1989.
Kadesch and Berg, Mol. Cell. Biol., 6:2593, 1986.
Karin et al., Mol. Cell. Biol., 7:606, 1987.
Karin et al., Mol. Cell. Biol., 7:606, 1987.
Katinka et al., Cell, 20:393, 1980.
Kawamoto et al., Mol. Cell. Biol., 8:267, 1988.
Kiledjian et al., Mol. Cell. Biol., 8:145, 1988.
Klamut et al., Mol. Cell. Biol., 10:193, 1990.
Klein et al., Nature, 327:70-73, 1987.
Koch et al., Mol. Cell. Biol., 9:303, 1989.
Kriegler and Botchan, In: Eukaryotic Viral Vectors, Gluzman (Ed.), Cold Spring Harbor: Cold Spring Harbor Laboratory, N Y, 1982.
Kriegler and Botchan, Mol. Cell. Biol., 3:325, 1983.
Kriegler et al., Cell, 38:483, 1984.
Kriegler et al., Cell, 53:45, 1988.
Kuhl et al., Cell, 50:1057, 1987.
Kunz et al., Nucl. Acids Res., 17:1121, 1989.
Larsen et al., Proc. Natl. Acad. Sci. USA., 83:8283, 1986.
Laspia et al., Cell, 59:283, 1989.
Latimer et al., Mol. Cell. Biol., 10:760, 1990.
Lee et al., Nature, 294:228, 1981.
Lee et al., Nucleic Acids Res., 12:4191-206, 1984.
Levinson et al., Nature, 295:79, 1982.
Lin et al., Mol. Cell. Biol., 10:850, 1990.
Luria et al., EMBO 1, 6:3307, 1987.
Lusky and Botchan, Proc. Natl. Acad. Sci. USA, 83:3609, 1986.
Lusky et al., Mol. Cell. Biol., 3:1108, 1983.
Majors and Varmus, Proc. Natl. Acad. Sci. USA, 80:5866, 1983.
Mann et al., Cell, 33:153-159, 1983.
McNeall et al., Gene, 76:81, 1989.
Miksicek et al., Cell, 46:203, 1986.
Mordacq and Linzer, Genes and Dev., 3:760, 1989.
Moreau et al., Nucl. Acids Res., 9:6047, 1981.
Muesing et al., Cell, 48:691, 1987.
Ng et al., Nuc. Acids Res., 17:601, 1989.
Nicolas and Rubinstein, In: Vectors: A survey of molecular cloning vectors and their uses, Rodriguez and Denhardt (Eds.), Stoneham: Butterworth, 494-513, 1988.
Nicolau and Sene, Biochim. Biophys. Acta, 721:185-190, 1982.
Ondek et al., EMBO 1, 6:1017, 1987.
Ornitz et al., Mol. Cell. Biol., 7:3466, 1987.
Palmiter et al., Cell, 29:701, 1982.
Palmiter et al., Nature, 300:611, 1982.
Paskind et al., Virology, 67:242-248, 1975.
Pech et al., Mol. Cell. Biol., 9:396, 1989.
Perez-Stable and Constantini, Mol. Cell. Biol., 10:1116, 1990.
Picard and Schaffner, Nature, 307:83, 1984.
Pinkert et al., Genes and Dev., 1:268, 1987.
Ponta et al., Proc. Natl. Acad. Sci. USA, 82:1020, 1985.
Porton et al., Mol. Cell. Biol., 10:1076, 1990.
Potter et al., Proc. Natl. Acad. Sci. USA, 81:7161-7165, 1984.
Queen and Baltimore, Cell, 35:741, 1983.
Quinn et al., Mol. Cell. Biol., 9:4713, 1989.
Redondo et al., Science, 247:1225, 1990.
Reisman and Rotter, Mol. Cell. Biol., 9:3571, 1989.
Remington's Pharmaceutical Sciences, 15^{th} ed., 1035-1038 and 1570-1580, Mack Publishing Company, P A, 1980.
Renan, Radiother. Oncol., 19:197-218, 1990.
Resendez Jr. et al., Mol. Cell. Biol., 8:4579, 1988.
Ridgeway, In: Vectors: A survey of molecular cloning vectors and their uses, Stoneham: Butterworth, 467-492, 1988.
Ripe et al., Mol. Cell. Biol., 9:2224, 1989.
Rippe et al., Mol. Cell Biol., 10:689-695, 1990.
Rittling et al., Nuc. Acids Res., 17:1619, 1989.
Rosen et al., Cell, 41:813, 1988.
Sakai et al., Genes and Dev., 2:1144, 1988.
Satake et al., J. Virology, 62:970, 1988.
Schaffner et al., J Mol. Biol., 201:81, 1988.
Searle et al., Mol. Cell. Biol., 5:1480, 1985.
Sharp and Marciniak, Cell, 59:229, 1989.
Shaul and Ben-Levy, EMBO J, 6:1913, 1987.
Sherman et al., Mol. Cell. Biol., 9:50, 1989.
Sleigh and Lockett, J EMBO, 4:3831, 1985.
Spalholz et al., Cell, 42:183, 1985.
Spandau and Lee, J Virology, 62:427, 1988.
Spandidos and Wilkie, EMBO 1, 2:1193, 1983.
Stephens and Hentschel, Biochem. 1, 248:1, 1987.
Stuart et al., Nature, 317:828, 1985.
Sullivan and Peterlin, Mol. Cell. Biol., 7:3315, 1987.
Swartzendruber and Lehman, J Cell. Physiology, 85:179, 1975.
Takebe et al., Mol. Cell. Biol., 8:466, 1988.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J Biol. Chem., 264:15160, 1989.
Temin, In: Gene Transfer, Kucherlapati (Ed.), NY, Plenum Press, 149-188, 1986.
Thiesen et al., J. Virology, 62:614, 1988.
Triesman, Cell, 42:889, 1985.
Tronche et al., Mol. Biol. Med., 7:173, 1990.
Trudel and Constantini, Genes and Dev. 6:954, 1987.
Tur-Kaspa et al., Mol. Cell Biol., 6:716-718, 1986.
Tyndell et al., Nuc. Acids. Res., 9:6231, 1981.
Vannice and Levinson, J. Virology, 62:1305, 1988.
Vasseur et al., Proc Natl. Acad. Sci. U.S.A., 77:1068, 1980.
Weber et al., Cell, 36:983, 1984.
Weinberger et al. Mol. Cell. Biol., 8:988, 1984.
Winoto and Baltimore, Cell, 59:649, 1989.
Wu and Wu, Biochemistry, 27:887-892, 1988.
Wu and Wu, J. Biol. Chem., 262:4429-4432, 1987.
Yang et al., Proc. Natl. Acad. Sci. USA, 87:9568-9572, 1990.
Yutzey et al. Mol. Cell. Biol., 9:1397, 1989.
Godschalk et al., Mutagenesis 25, 149-154, 2009.
Atkins et al., Cancer Res. 69, 1553-1560, 2009.
Jurk et al., Nat. Commun. 6, 6818, 2015.
Gregory & Houghton, Cancer Research 71, 2411-2416, 2011.
Nozawa et al., Proc. Natl. Acad. Sci. 103, 12493-12498, 2006.
Albelda et al., Cancer Cell 16, 183-194, 2009.
Thacker et al., J. Immunol. 184, 3284-3297, 2010.
Szczerba et al., Nature 566, 553-557, 2019.
Wculek & Malanchi, Nature 528, 413-417, 2015.
Brinkmann et al., Science 303, 1532-1535, 2004.
Papayannopoulos, V., Nature Reviews Immunology 18, 134-147, 2018.
De Larco et al., Clin. Cancer Res. 10, 4895-4900, 2004.
Demers et al., Proc. Natl. Acad. Sci. 109, 13076-13081, 2012.
Giannias et al., J. Clin. Invest. 123, 3446-3458, 2013.
Mitchell et al., Proc. Natl. Acad. Sci. 111, 930-935, 2014.
Li et al, J. Control. Release 228, 38-47, 2016.
Pedruzzi et al., Br. J. Haematol. 117, 719-26, 2002.
Lievin-Le Moal et al., Infect. Immun. 80, 1891-1899, 2012.

1. A method inhibiting cancer in a subject comprising administering to said subject a first dose of a neutrophil engineered to express TNF-related apoptosis inducing ligand (TRAIL) fused to a positively-charged peptide or protein segment.

2. The method of claim 1, wherein said engineered neutrophil has been transformed with an exogenous mRNA encoding the TRAIL fusion.

3. The method of claim 1, wherein said engineered neutrophil has been transformed with an expression vector encoding the TRAIL fusion.

4. The method of claim 3, wherein said expression vector is a viral expression vector.

5. The method of claim 3, wherein said expression vector is a non-viral expression vector.

6. The method of claim 1, wherein said engineered neutrophil is administered systemically.

7. The method of claim 1, wherein said engineered neutrophil is administered local, such as into a tumor bed or into tumor vasculature, or regional to a cancer site.

8. (canceled)

9. The method of claim 1, further comprising administering to said subject a second dose of said engineered neutrophil.

10. The method of claim 1, wherein said positively-charged protein or peptide is a protein with electrostatic charge ranging from +4 to +24.

11. (canceled)

12. The method of claim 1, further comprising administering to said subject a second cancer therapy.

13. The method of claim 12, wherein said second cancer therapy is chemotherapy, radiotherapy, immunotherapy, surgery or hormonal therapy.

14. The method of claim 1, wherein said neutrophil is autologous to said subject.

15. The method of claim 1, wherein said neutrophil is not autologous to said subject.

16. The method of claim 1, wherein said subject is a non-human animal.

17. The method of claim 1, wherein said subject is a human.

18. The method of claim 1, wherein said cancer is recurrent, metastatic and/or or drug resistant.

19. The method of claim 1, wherein inhibiting cancer comprises inducing apoptosis in cells of said cancer.

20. The method of claim 1, further comprising co-administering one or more agents that promote netosis, such as fmlp or IL-8, which may be delivered liposomes or hydrogels, or in pH sensitive liposomes or hydrogels.

21. A neutrophil engineered to express TNF-related apoptosis inducing ligand (TRAIL) fused to a positively-charged peptide or protein segment.

22-30. (canceled)

31. A method inhibiting cancer in a subject comprising transfecting a pluripotent stem cell or a neutrophil in said cell with a construct that expresses TNF-related apoptosis inducing ligand (TRAIL) fused to a positively-charged peptide or protein segment.

32. (canceled)


SEQUENCE LISTING (1 sequence)

SEQ ID NO: 1 - DNA, 78 bp, Artificial sequence
  Synthetic oligonucleotide
gggtctgggtaggaacccagaaacctctgacagaagaatcgaaattaatacgactcactatagggagacccaagctgg